(19) United States
(12) Patent Application Publication
(10) Pub. No.: US 2002/0001577 A1
(43) Pub. Date: Jan. 3, 2002

(54) Use of cells with the ability to generate excitation within a cell preparation

(76) Inventors: Axel Haverich, Isernhagen (DE); Arjang Ruhparwar, Isernhagen (DE)

Correspondence Address: McGuireWoods, Suite 1800, 1750 Tysons Boulevard, McLean, VA, 22102, US

(21) Appl. No.: 09/761,863

(22) Filed: Jan. 18, 2001

(30) Foreign Application Priority Data
Jun. 30, 2000 (DE) 100 31 138.5-41

(51) Int. Cl. C12N 5/08; A61K 45/00

(52) U.S. Cl. 424/93.7; 435/366

(57) ABSTRACT

Cells with the capability to generate excitation in a heart muscle tissue are used in this invention as biological cardiac pacemakers. That is done by suspending the dissociated cells and injecting them at a suitable position into the heart of a patient. The suspension can contain medicinal additives.

### BACKGROUND OF THE INVENTION

1. Field of the Invention

The invention concerns the use of cells with the ability to generate excitation in heart muscle tissue for therapeutic purposes, and a cell preparation suitable for such use.

The concept of using cells deliberately inserted into the heart from outside rather than an artificial cardiac pacemaker is new.

2. Background Description

A heart naturally has, in certain spatially limited regions, muscle cells specialized particularly for producing excitation. These cells with cardiac pacemaker potential appear in, among other sites, the sinus node, the primary physiological cardiac pacemaker, in which excitation occurs autonomically and rhythmically. They have frequencies greater than the inherent frequencies of the other segments of the autonomic excitatory system. Such cells also appear in deeper segments of the heart and in the AV (atrioventricular) nodes, but always with lower inherent frequency.

If the sinus node fails, the AV nodes or deeper segments of the excitatory conduction system can take over the function of a physiological cardiac pacemaker.

In case of persistent trouble in the physiological cardiac pacemaker system, finally, an artificial cardiac pacemaker becomes necessary.

Artificial cardiac pacemakers are devices which act, by electrical stimulation, as pulse generators for regular cardiac activity. These pulse generators are available as transportable extracorporeal devices, or as small implantable devices. The electrical impulses are transmitted to the heart muscle through an electrode.

Although cardiac pacemaker implantation is now considered routine surgery, it involves a major and critical operation with substantial stresses for the patient. There can be complications with use of cardiac pacemakers, such as in the vicinity of strong magnetic fields. The patient is also advised that the batteries work absolutely reliably.

### SUMMARY OF THE INVENTION

Thus the objective of the invention is to make available a cardiac pacemaker which is more easily implantable, with low maintenance, as well as a cardiac pacemaker for cases in which use of an artificial cardiac pacemaker is impossible.

The invention considers that this objective is attained by using cells with the ability for rhythmic contraction, thus linked to generation of excitation in cardiac muscle tissue, in a cell preparation intended to be inserted at least once in a suitable position into the heart of a patient, as a cardiac pacemaker.

The invention is based on the recognition that suitably selected and/or prepared living cells placed in the heart of a patient can exert a cardiac pacemaker function there. The cells to be added as the cardiac pacemaker interact with the cells around the site of administration and stimulate the entire cardiac muscle tissue. The administration can be done in all the segments of the heart which contain cardiac muscle cells.

The cells used preferably have a higher inherent frequency than the cells in the vicinity of the site at which the cell preparation is to be applied.

The invention also covers a cell preparation to be made available, which is usable as a “biological cardiac pacemaker” in the sense presented above.

### BRIEF DESCRIPTION OF THE DRAWINGS

The foregoing and other objects, aspects and advantages will be better understood from the following detailed description of a preferred embodiment of the invention with reference to the drawings, in which:

FIG. 1A shows HE staining (200×) of a CXMD dog heart three weeks after injection of fetal atrial cardiomyocytes (FCM). The FCM (arrow points) lie in and around the injection channel. Points of accumulated dystrophic calcifications were found (arrow). RCM: receptor cardiomyocytes;

FIG. 1B shows Dystrophin staining (630×). Only FCM dystrophinpositive (dark). No expression in RCM.;

FIG. 1C shows Connexin 43 staining (630×). Strong color pattern at the cell contacts between RCM and FCM (arrow).

### DETAILED DESCRIPTION OF A PREFERRED EMBODIMENT OF THE INVENTION

It is important for the invention that the cells used can take on their function as “biological cardiac pacemaker” immediately after administration. Therefore the cells may be appropriately cultured or prepared. The cells in the cell preparation comprise adult, fetal or neonatal cardiomyocytes, preferably atrial cells and/or sinus node cells, somatic stem cells developed into cardiomyocytes or precursors of them, or other cells cultured or adapted for the ability to generate rhythmic contraction or cardiologic excitation.

The cardiomyocytes will often be allogeneic cells. To avoid rejection reactions, these cells can be administered along with immunosuppressants. Cyclosporines and corticosteroids are suitable immunosuppressants.

Other cells to be used according to the invention are cardiomyocytes or (immediate) precursors of them developed from somatic stem cells. For this purpose, somatic stem cells, such as bone marrow cells, from the patient to receive the heart pacemaker, are cultured in vitro into cardiomyocytes. For example, a process which can be used for this purpose is that presented in detail in the publication “Cardiomyocytes can be generated from marrow stromal cells in vitro”, Shinji Makino et al. in: The Journal of Clinical Investigation, March 1999, Volume 103, No. 5, pages 697-703. Reference is expressly made here to its content. Only the cells preferably [sic] transformed completely in vitro are used as biological cardiac pacemakers.

Furthermore, cells adapted for the application are suitable according to the invention. They are likewise prepared in vitro. “Adapted cells” means, for example, autologous myocytes conditioned by coupling with allogeneic or xenogeneic cardiomyocytes. Alternatively, allogeneic cardiomyocytes modified by genetic engineering can, among others, be used.

Another possibility for getting cells usable in this invention comprises controlled proliferation of adult autologous cardiomyocytes. That can be accomplished, for instance, by adding mediators which promote regeneration and growth, and/or excitation mediators [see note 1]. In the cell preparation, the cells occur dissociated in the simplest form in a suspension, with which the suspending agent may be a nutrient solution or a physiological solution. In the simplest case, the cells can be dissociated with collagenase, for instance, and suspended in the desired medium.

The suspension can also be thixotropic or viscous, principally to retain it better at the site of administration. In addition, the suspension may contain other cells; adjuvants (e.g., CAMP [cyclic adenosine monophosphate]); medications, especially supportive cardiac medications, antibiotics, immunosuppressants, and the like, and/or mediators. It is currently considered particularly advantageous for the suspension to contain cyclic AMP.

The cell preparation according to the invention is a “biological cardiac pacemaker” which can be “implanted” without complication. Use of the “biological cardiac pacemaker” according to the invention opens up new perspectives for treatment of cardiac rhythm disturbances and AV [atrioventricular] block. The invention can be particularly advantageous for children and premature infants with congenital AV block, who are too small to be treated with artificial cardiac pacemakers.

In the following, an animal experiment is described as a preliminary experiment on the mode of action of the new “biological cardiac pacemaker”. In the investigations described therein, the usability of the cells in the sense of the invention was tested.

Exemplary Investigation of the Functioning of CXMD Dogs

The studies described below showed that transplanted cells used as cardiac pacemakers provide a coherent contribution to contraction and electrical excitation of the host myocardium in vivo. The object of the investigations was to show that, by transplantation of cardiomyocytes with higher intrinsic rhythmic pulse rate into the myocardium of the left ventricle, those cells could function as ectopic cardiac pacemakers.

For this purpose, fetal dog atrial cardiomyocytes, including sinus node cells, were introduced into the free wall of the left ventricles of mature CXMD (canine X-linked muscular dystrophy) dogs. These dogs cannot express dystrophin in either cardiac or skeletal muscle. The transplanted cells were identified by their immunoreactivity to dystrophin, indicating their survival and morphologic integration into the receptor heart. After catheter ablation of the atrioventricular (AV) node, electrophysiological recordings provided proof of the ability of the implanted cells to function as cardiac pacemakers, because most of the cardiac pacemaker activity was derived from the labeled transplant region. This effect was not observed in the control group, in which fetal skin fibroblasts were transplanted into the heart.

These results show electrical and mechanical coupling between donor and receptor cells, and open up new therapeutic potentials for treatment of congenital and acquired atrioventricular block.

The dystrophin gene product was used to follow the fate of the cardiomyocytes used. Immunohistologic analyses with anti-dystrophin antibodies showed the presence of dystrophin-positive donor cardiomyocytes 3 weeks after implantation in the dystrophin-negative receptor myocardium, in a limited region about the site of implantation. In contrast, no dystrophin-positive cardiomyocytes were found in the control receptor myocardium.

The treated regions were also examined for expression of connexin 43. This molecule is a major component within the open cell contacts in the heart, which makes transmission of electrical excitation possible, thus passing on the contraction from cell to cell. The connexin 43 immunoreactivity was visible at the linkage complexes between donor and receptor c ells, indicating a morphologic coupling as the result of bridging over the space.

The dogs were subjected to a detailed electrophysiological examination three to four weeks after the transplantation. All the examinations were done with X-ray control. First, a completely temperature-controlled radio-frequency catheter ablation of the AV node was done. Then a replacement rhythm with narrow/dense QRS complexes (average rate 45+ beats per minute) was observed. Five to thirty minutes after ablation of the AV node, a faster replacement rhythm than the preceding one, with broad QRS complexes (right Tawara-Schenkel block configuration) appeared in all the dogs which had been transplanted with fetal cardiomyocytes. It did not appear in any of the control dogs, in spite of provocative tests with atropine, orciprenaline and rapid ventricular pacing after a waiting period of 50 minutes.

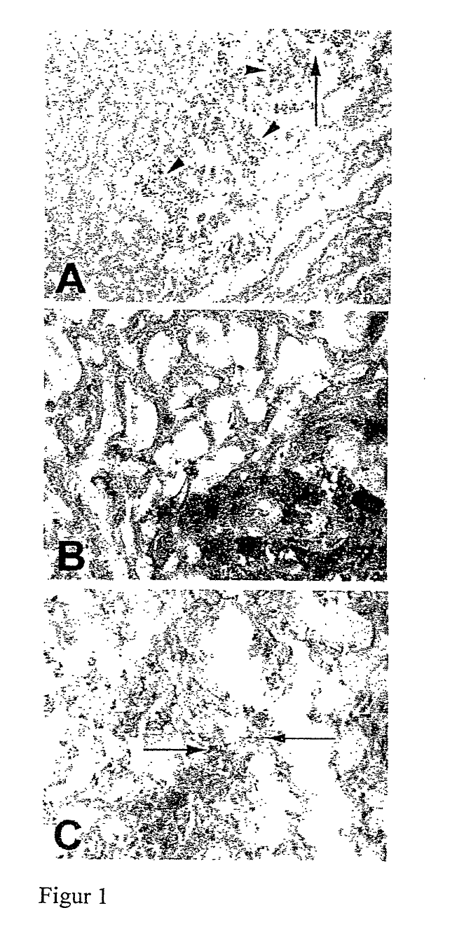

To be able to evaluate the origin of the faster replacement rhythm more accurately, a detailed recording was done in the left ventricle with a controllable 7F recording catheter. An impulse determination analysis with agreement of 6 of 6 surface electrocardiograms (EKGs), a QA pattern in the unipolar electrogram, and an earliest starting point of 10 ms before the QRS onet in the bipolar electrogram were obtained in the endocardial region in very close association with the labeled epicardial transplantation site in the left ventricle (FIG. 1B).

This intrinsic replacement rhythm was stable after removal of the pacemaking catheter and over a follow-up time of 45 minutes. The recovery time of the replacement rhythm after 2 minutes of rapid ventricular pacemaking (150 beats/minute) was 1 minute.

The results prove that dogs transplanted with fetal atrial cardiomyocytes develop a replacement rhythm which propagates from the site of myocyte injection into the myocardium of the left chamber. This shows functional coupling between donor and receptor cells which makes it possible for the cells used to develop and maintain a stable ventricular replacement rhythm. The site of origin of the ventricular replacement rhythm was identified and classified by analysis of the activation and impulse site.

Methods

Cell Recovery.

Fetuses from the third trimester were used. They were carried to term and delivered by Caesarian section. Skin segments (control group) and atria, including the sinus nodes, were dissected out and dissociated in 0.1% Collagenase A solution (Boehringer Mannheim). Cardiomyocytes and skin fibroblasts were isolated. 2×10⁶ cardiomyocytes were injected into the free walls of the left ventricles of mature CXMD dogs by anterolateral thoracotomy. An epimyocardial pacemaker system with a rate of at least 30/minute (VVI mode) was also implanted at the same time to assure survival of the AV node ablation. The same procedure was done in the control group, using fetal skin fibroblasts. For fluoroscopic detection, the injection sites were marked with Prolen [see note 2] and titanium clips were applied. Daily administration of Cyclosporin (15 mg/kg) and corticosteroids (2.5 mg/kg) provided reliable immune suppression.

Immunohistologic Analysis.

The hearts were dissected out and shock-frozen in liquid nitrogen. The usual hematoxylin and eosin stains were done on serial sections. The sections were fixed in acetone (10 minutes at −20° C., blocked with normal rabbit serum (room temperature, 15 minutes), and then incubated with the primary monoclonal mouse antibodies. Anti-dystrophin clone NCL-Dys 1 1:5 (Novocastra Lab., UK) and anti-connexin 43 clone MAB 3068 1:40, were each incubated for 1 hour at 37° C. After incubation of the biotinylated secondary antibodies, detection was accomplished with a streptavidin-alkaline phosphatase complex. The substrate was neofuchsin, with hemalum as the counterstain. Suitable positive and negative staining was done for each antibody.

Electrophysiological Investigations and Radio-frequency Catheter Ablation.

Three to four weeks after the transplantation, the dogs were anesthetized and introducer sheaths were inserted into the right femoral artery and vein. A 6F Hexapolar catheter was placed at the region of the bundle of His, and a 7F controllable ablation catheter was positioned at the position of the presumed compact AV node, steered with anatomic and electrophysiological guidance. After one to three temperature-controlled radiofrequency treatments, complete AV block was induced. Activations and impulse recordings from the left ventricle were done with the 7F controllable recording-mapping catheter.

Having thus described our invention, what we claim as new and desire to secure by Letters Patent is as follows:

1. A method of treating heart muscle tissue, comprising the steps of obtaining a cell preparation which includes cells that produce excitation in heart muscle tissue; and
inserting said cell preparation in to the heart of patient to excite said heart muscle tissue.

2. The method of claim 1, wherein the cells have a higher inherent frequency than cells in at a site of insertion used in said insertion step.

3. The method of claim 1, wherein the cells are selected from the group consisting of adult, fetal, or neonatal cardiomyocytes, somatic stem cells developed into cardiomyocytes or precursors of said somatic stem cells, and other cells which have the ability to generate a rhythmic contraction or cardiologic excitation.

4. A cell preparation comprising adult, fetal or neonatal cardiomyocytes, somatic stem cells developed into cardiomyocytes or precursors of them, or other cells cultured or adapted for the ability to generate rhythmic contraction or cardiologic excitation, dissociated in a suspension.

5. The cell preparation according to claim 4 wherein the suspension is thixotropic or viscous.

6. The cell preparation according to claim 4, wherein the suspension contains ingredients selected from the group consisting of other cells, adjuvants, medications and/or mediators.

7. The cell preparation according to claim 6, further comprising cyclic adenosine monophosphate.
